(19) United States
(12) Patent Application Publication
(10) Pub. No.: US 2008/0287948 A1
(43) Pub. Date: Nov. 20, 2008

(54) Electrosurgical system

(75) Inventors: Michael D. Newton, Wales (GB); Richard J. Curtis, Newport (GB)

Correspondence Address: NIXON & VANDERHYE, PC, 901 NORTH GLEBE ROAD, 11TH FLOOR, ARLINGTON, VA, 22203, US

(73) Assignee: GYRUS MEDICAL LIMITED, Cardiff (GB)

(21) Appl. No.: 12/149,528

(22) Filed: May 2, 2008

Related U.S. Application Data
(60) Provisional application No. 60/924,961, filed on Jun. 6, 2007.

(30) Foreign Application Priority Data
May 4, 2007 (GB) 0708783.6

(51) Int. Cl. A61B 18/18

(52) U.S. Cl. 606/50

(57) ABSTRACT

An electrosurgical system includes a generator for generating radio frequency power, an electrosurgical instrument including at least first and second bipolar electrodes carried on the instrument, and a monopolar patient return electrode separate from the instrument. The generator comprises a source of radio frequency (RF) power, and has a first supply state in which the RF waveform is supplied between the first and second bipolar electrodes of the electrosurgical instrument, and a second supply state in which the RF waveform is supplied between at least one of the first and second bipolar electrodes and the monopolar patient return electrode. A controller is operable to control the generator such that, in at least one mode of the generator, a feeding means is adapted to alternate between the first and second supply states to supply an alternating signal.

### BACKGROUND OF THE INVENTION

This invention relates to an electrosurgical system including a bipolar electrosurgical instrument for use in the treatment of tissue.

Both monopolar and bipolar electrosurgery are well-established techniques. In monopolar electrosurgery, an electrosurgical instrument has a single electrode and a patient return plate is attached to the patient well away from the electrosurgical instrument. The electrosurgical current flows from the electrode through the patient to the return plate.

In bipolar electrosurgery, the electrosurgical instrument includes spaced first and second electrodes, and there is no patient return plate. The current flows from one electrode through the patient to the other, and so the current flow is kept to a much more localised area.

Both monopolar and bipolar electrosurgery are known to have certain advantages and disadvantages. Monopolar electrosurgery is known to produce very effective tissue coagulation, but there is always the danger of stray current paths causing the unwanted treatment of tissue spaced from the monopolar electrode. Burns to the patient in the area of the return plate have also been known. Bipolar electrosurgery is generally considered to be a safer option, as the current is constrained within a smaller area, but it is sometimes difficult to obtain as thorough a coagulation effect with a bipolar instrument.

For this reason perhaps, there have been previous attempts to provide the option of either monopolar or bipolar electrosurgery from a single generator. The prior art is full of examples of generators in which both a monopolar and a bipolar instrument can be connected to the generator, with some form of switch to select which one of the instruments is to be activated at any one time. Examples include U.S. Pat. Nos. 4,171,700, 4,244,371, 4,559,943, 5,951,545 and 6,113,596. U.S. Pat. No. 5,472,442 is different in that a single instrument can be used in either a monopolar or bipolar mode, but once again a choice must be made as to which one of monopolar or bipolar modes is to be activated at any one time.

### SUMMARY OF THE INVENTION

The present invention attempts to provide an easy to use electrosurgical system enjoying the benefits of both monopolar and bipolar electrosurgery. Accordingly, an electrosurgical system is provided including a generator for generating radio frequency (RF) power, an electrosurgical instrument including at least first and second bipolar electrodes carried on the instrument, and a monopolar patient return electrode separate from the instrument, wherein the generator comprises at least one source of RF power and a plurality of outputs connected to the electrodes, the generator being adapted to operate in a first supply state in which an RF output waveform is delivered between the first and second bipolar electrodes via the output lines, and in a second supply state in which an RF output waveform is delivered between (a) at least one of the first and second bipolar electrodes and (b) the monopolar patient return electrode via the output lines, which operation, in at least one mode of the generator, includes continuously alternating between the first supply state and the second supply state whereby combined bipolar and monopolar RF energy delivery is obtained.

The generator effectively delivers an RF waveform in both the first and second supply states. In one arrangement, the generator includes first and second sources of radio frequency (RF) power, the first source being connected to deliver an RF waveform in the first supply state, and the second source being connected to deliver an RF waveform in the second supply state. In a preferred generator, a feeding means is adapted to supply an RF waveform between the bipolar electrodes simultaneously with an RF waveform being supplied between one bipolar electrode and the patient return electrode. Alternatively, the feeding means is adapted to supply RF waveforms from at least one of the first and second sources discontinuously, with one or both of the sources being switched in and out of connection with the electrodes. In one arrangement, the feeding means is adapted to switch in and out the connection of the first source to deliver the RF waveform in the first supply state discontinuously.

In accordance with the invention, the feeding means is adapted to alternate between the first and second supply states, either with or without gaps therebetween. In this arrangement there is a regular switching between the first supply state, in which the RF waveform is supplied “bipolar” mode, and the second supply state, in which the RF waveform is supplied in “monopolar” mode. As the regular switching between the first and second states takes place at a high frequency, typically between 5 and 100 Hz, the overall effect is a blend of monopolar and bipolar electrosurgery delivered substantially simultaneously.

The “first duty cycle” is defined as that part of the overall signal that is delivered in the first supply state. Similarly, the “second duty cycle” is defined as that part of the overall signal that is delivered in the second supply state. In general terms, the first duty cycle is the proportion of the signal that is delivered in the “bipolar” mode, and the second duty cycle is the proportion of the signal that is delivered in the “monopolar” mode. If a single source is provided and switched between the electrodes, then a first duty cycle of 30% would see the waveform delivered in bipolar mode for 30% of the time and in monopolar mode for 70% of the time (if there were no gaps between the various parts of the signals). A first duty cycle of 30% and a second duty cycle of 50% would see a gap between the bipolar and monopolar parts of the signal, the gap constituting 20% of the overall cycle.

In one convenient arrangement, both the first and second duty cycles are constant at 50%, thereby providing equal periods for both bipolar and monopolar modes. In an alternative arrangement, at least one duty cycle is not constant, and there is adjustment means, operable by the user of the electrosurgical system, for changing at least one duty cycle. Typically, the adjustment means is operable by the user of the electrosurgical system to change the at least one duty cycle between a plurality of preset settings. In this way, the user can select various settings for the duty cycle, for example mostly bipolar, mostly monopolar, equal amounts of bipolar and monopolar etc. If desired, the user could be permitted to use the electrosurgical instrument entirely in bipolar or monopolar mode, if required.

Alternatively, the electrosurgical system includes means for measuring a parameter associated with the electrosurgical procedure, the controller adjusting at least one duty cycle automatically in response to the measured parameter. In this way, the electrosurgical system adjusts itself dynamically in response to different operating conditions, selecting greater or lesser proportions of the bipolar and monopolar modes respectively, as required for effective operation. Conveniently, the measured parameter is the impedance measured between two of the electrodes. This could be the impedance between the two bipolar electrodes, or alternatively one of the bipolar electrodes and the patient return plate. Thus, when the measured impedance is low, indicating a relatively fluid surgical environment associated with bleeding tissue, the electrosurgical system could increase the proportion of the monopolar signal applied to the tissue, as this is recognized as providing effective coagulating power. Conversely, when the measured impedance is higher, indicating a relatively dry surgical environment, the electrosurgical system could increase the proportion of bipolar signal applied to the tissue, in order to maximise patient safety.

In another convenient arrangement, the feeding means operates such that at least one duty cycle varies according to a predetermined progression. This provides a dynamically changing electrosurgical signal, without the user selecting different operating settings, or the system performing dynamic measurement of operating parameters. For example, experience could show that the most effective tissue coagulating waveform for a particular tissue or vessel type is a particular combination of bipolar and monopolar signals, changing over time. This could be preprogrammed into the electrosurgical generator, such that it is automatically performed without the need for any additional intervention from the user. Conceivably, the predetermined progression is such that at least one duty cycle increases or alternatively decreases with time. Alternatively, the feeding means operates such that there is a first period during which the duty cycle is constant, followed by a second period in which at least one duty cycle varies according to a predetermined progression. Different predetermined progressions of duty cycle may be appropriate for different types of tissue, or for different surgical procedures, as will be readily established by users of the electrosurgical system.

The monopolar patient return electrode is described as being separate from the instrument. This is to say that the monopolar patient return electrode is designed to be attached to the patient at a location remote from the area where the instrument is in contact with the patient. Conceivably, the patient return electrode could still be supplied together with the electrosurgical instrument, and may even be physically attached thereto, for example by means of a long cord or tie. The description of the monopolar patient return electrode as being “separate” refers to its remote location on the patient, as opposed to any lack of connection with the electrosurgical instrument.

Conceivably, a characteristic of the RF waveform is different during the first duty cycle as compared to the second duty cycle. For example, the power of the RF waveform may be different during the bipolar mode as compared with the power during the monopolar mode. Similarly, the voltage of the RF waveform, the current of the RF waveform, or the frequency of the RF waveform could be different for the bipolar signals as opposed to the monopolar signals.

The electrosurgical system according to the present invention is primarily concerned with the effective coagulation of tissue, but the electrosurgical system can also be employed to cut or vaporise tissue. In a convenient arrangement, the electrosurgical instrument includes at least a third electrode, and the generator is adapted, in an alternative mode of operation, to supply a cutting RF waveform between the third electrode and one or both of the first and second electrodes. Thus, the instrument can be employed to cut or vaporise tissue, and then coagulate tissue in either a bipolar or monopolar mode, or a combination of bipolar and monopolar modes.

The invention further resides in an electrosurgical generator for generating radio frequency power, the generator including a bipolar output for an electrosurgical instrument including at least two output lines for bipolar electrodes carried on the instrument, and a monopolar output for a monopolar patient return electrode separate from the instrument; the generator comprising one source of radio frequency (RF) power, and having a first supply state in which the RF waveform is supplied to the bipolar output between the two output lines, and a second supply state in which the RF waveform is supplied between one or both of the two output lines of the bipolar output and the monopolar output, and a controller operable to control the generator such that, in at least one mode of the generator, a feeding means is adapted to alternate between the first and second supply states to produce an alternating signal.

The invention will be described in more detail, by way of example only, with reference to the accompanying drawings.

### DESCRIPTION OF THE DRAWINGS

In the drawings:

FIG. 1 is a schematic sectional view of an electrosurgical system according to the invention;

FIG. 2 is a schematic diagram of one embodiment of an electrosurgical system;

FIG. 3 is a schematic diagram of an electrosurgical system according to the invention;

FIGS. 4 to 6 are schematic diagrams showing the electrosurgical system of FIG. 3 in different modes of operation;

FIGS. 7a to 7d are schematic cross-sectional views showing the effect on tissue of different modes of operation of the electrosurgical system of FIGS. 2 to 6;

FIGS. 8a to 8e are schematic diagrams showing different outputs of the electrosurgical system of FIGS. 2 to 6;

FIG. 9 is a schematic diagram showing a variation of the electrosurgical system of FIG. 3 in accordance with an alternative embodiment of the invention;

FIGS. 10a and 10b are schematic diagrams showing further different outputs of the electrosurgical system of FIGS. 2 to 6;

FIGS. 11a to 11c are schematic diagrams showing further different outputs of the electrosurgical system of FIGS. 2 to 6; and

FIG. 12 is a schematic perspective view of an instrument useable as part of the electrosurgical system of FIG. 1.

### DETAILED DESCRIPTION OF THE INVENTION

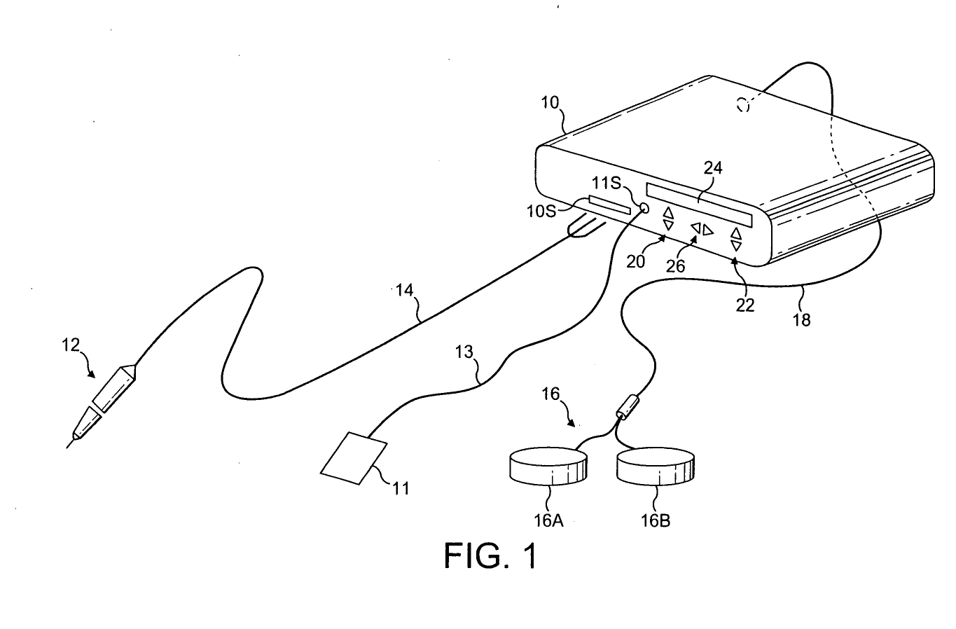

Referring to FIG. 1, a generator 10 has an output socket 10S providing a radio frequency (RF) output for an instrument 12 via a connection cord 14. An output socket 11S provides a connection for a patient return plate 11, via cord 13. Activation of the generator may be performed from the instrument 12 via a control connection in cord 14 or by means of a footswitch unit 16, as shown, connected separately to the rear of the generator 10 by a footswitch connection cord 18. In the illustrated embodiment, footswitch unit 16 has two footswitches 16A and 16B for selecting a coagulation mode and a cutting mode of the generator respectively. The generator front panel has push buttons 20 and 22 for respectively setting coagulation and cutting power levels, which are indicated in a display 24. Push buttons 26 are provided as an alternative means for selection between coagulation and cutting modes.

Referring to FIG. 2, generator 10 has a first RF power source 1 and a second RF power source 2. Instrument 12 includes bipolar electrodes 3A and 3B, and power source 1 is connected between electrodes 3A and 3B via lines 4A and 4B. Power source 2 is connected between line 4B (and hence electrode 3B) and the patient return plate 11 (via cord 13). A combining/protecting circuit such as a filter/adder circuit 5 is located between each power source and the line 3B to prevent signals from one power source being fed back to the other power source. In this way, one power source is prevented from causing damage to the other power source, and the signals therefrom are fed solely to the electrodes 3A and 3B, or the patient plate 11.

The operation of the electrosurgical system of FIG. 2 is as follows. When the footswitch 16A is activated to select the coagulation mode of the generator, power source 1 supplies an RF signal between bipolar electrodes 3A and 3B, while power source 2 supplies an RF signal between electrode 3B and the patient return plate 11. Thus the tissue 8 simultaneously receives both a bipolar tissue effect by virtue of electrodes 3A and 3B, and a monopolar tissue effect by virtue of electrode 3B and patient return plate 11. The power levels of sources 1 and 2 may be set at different levels, as is required for bipolar and monopolar signals respectively. Indeed, the power levels of power sources 1 and 2 may be adjusted, manually or automatically, in order to vary the tissue effect achieved by the electrosurgical system.

Alternatively or additionally, a feeding means is provided, adapted to switch in and out the connection of the second source to deliver the RF waveform in the second supply state discontinuously. In this way, the generator can supply a number of different signals, including but not limited to the following;

i) simultaneous continuous signals from the first and second sources;

ii) a continuous signal from the first source, with an intermittent signal from the second source;

iii) a continuous signal from the second source, with an intermittent signal from the first source;

iv) alternate signals from the first and second sources, in a continuously alternating fashion; and

v) intermittent signals from both the first and second sources, with gaps therebetween.

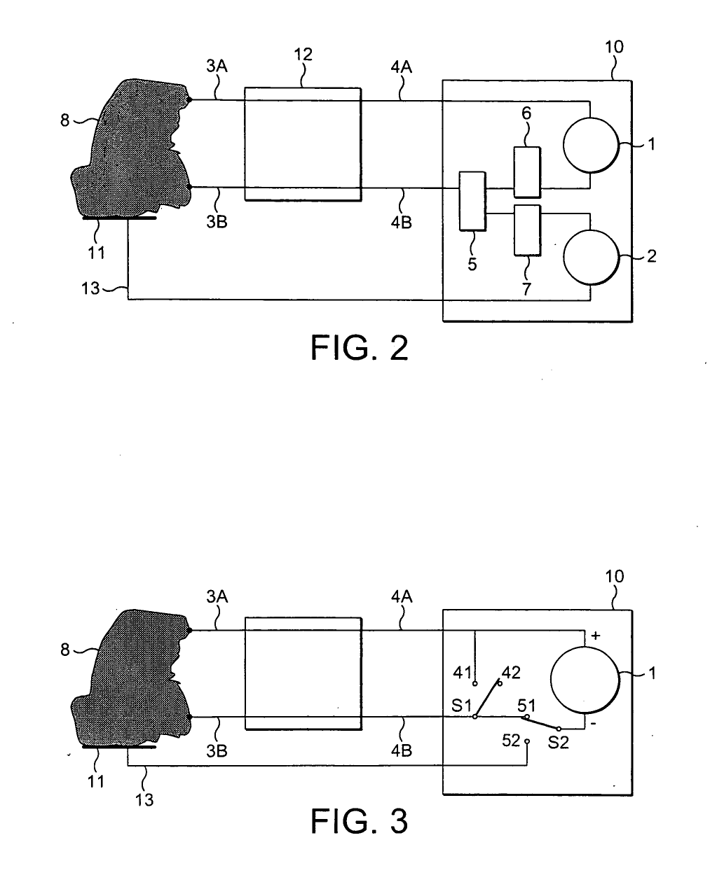

In this embodiment the switching is carried out by optional switching circuits 6 and 7 as the feeding means. Switching circuit 6 allows the signal from power source 1 to be optionally switched between connected and unconnected conditions with respect to output lines 4A and 4B. Similarly, switching circuit 7 allows the signal from power source 2 to be optionally switched between connected and unconnected conditions with respect to output lines 4B and 13. In this way, various combinations of simultaneous or sequential bipolar and monopolar signals can be applied to the tissue 8, as will be further described in more detail with respect to FIGS. 3 to 8.

FIG. 3 shows an embodiment in accordance with the invention in which the generator 10 has only a single RF power source 1. Power source 1 is connected to line 4A and hence bipolar electrode 3A, and also to line 4B via switches S1 and S2. Switches S1 and S2 are high-speed transistor switches, capable of switching between two alternate positions many times per second. Switch S1 is switched between two positions, a first position 41 in which lines 4A and 4B are connected, and a second position 42 in which they are separate. Switch S2 is also switched between two alternate positions, a first position 51 in which the power source 1 is connected to line 4B and a second position 52 in which the power source 1 is connected to cord 13 and hence the patient return plate 11.

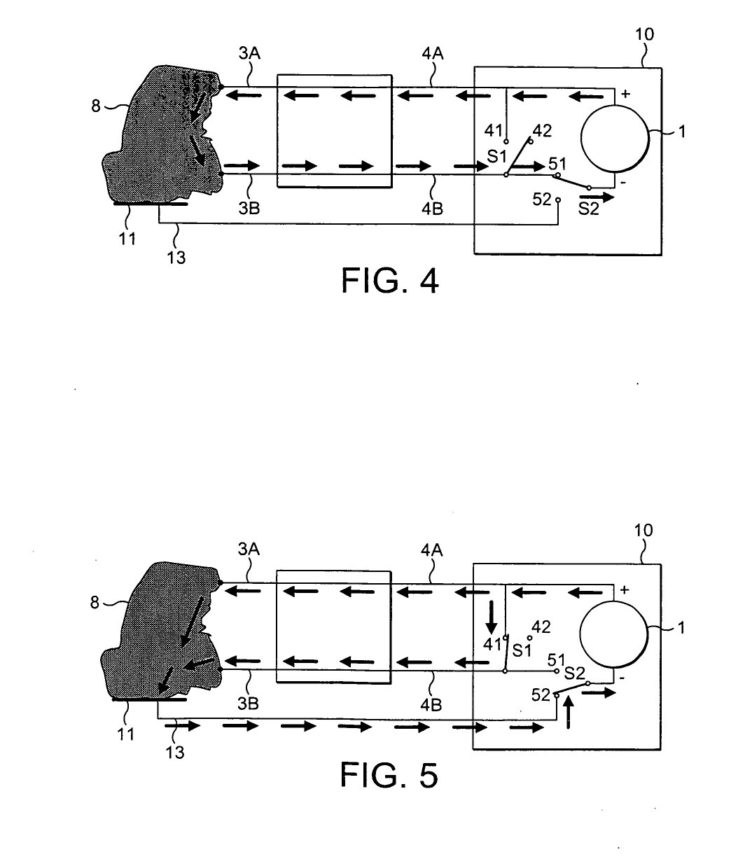

Switches S1 and S2 operate in tandem. FIG. 4 shows the situation when switch S2 is in its first position 51 and switch S1 is in its second position 42. In this arrangement the power source 1 is disconnected from the patient return plate 11 and connected across the bipolar electrodes 3A and 3B. This is the first supply state in which the RF waveform is supplied between the bipolar electrodes to provide a “bipolar” mode. FIG. 5 shows the opposite situation when switch S1 is in its first position 41 and switch S2 is in its second position 52. In this arrangement the lines 4A and 4B and hence the bipolar electrodes 3A and 3B are shorted together, and the power source is connected between these shorted electrodes and the patient return plate 11. This is the second supply state in which the RF waveform is supplied between one or both of the bipolar electrodes and the patient return electrode to provide a “monopolar” mode. The switches alternate in tandem between these two positions at a frequency of between 5 and 100 Hz to provide a continuous rapid alternation between the bipolar and monopolar modes. Thus the tissue effect achieved in the tissue 8 in the region of the electrodes 3A and 3B is a combination of bipolar and monopolar energy, with a greater depth of tissue coagulation than would be achieved by bipolar energy alone.

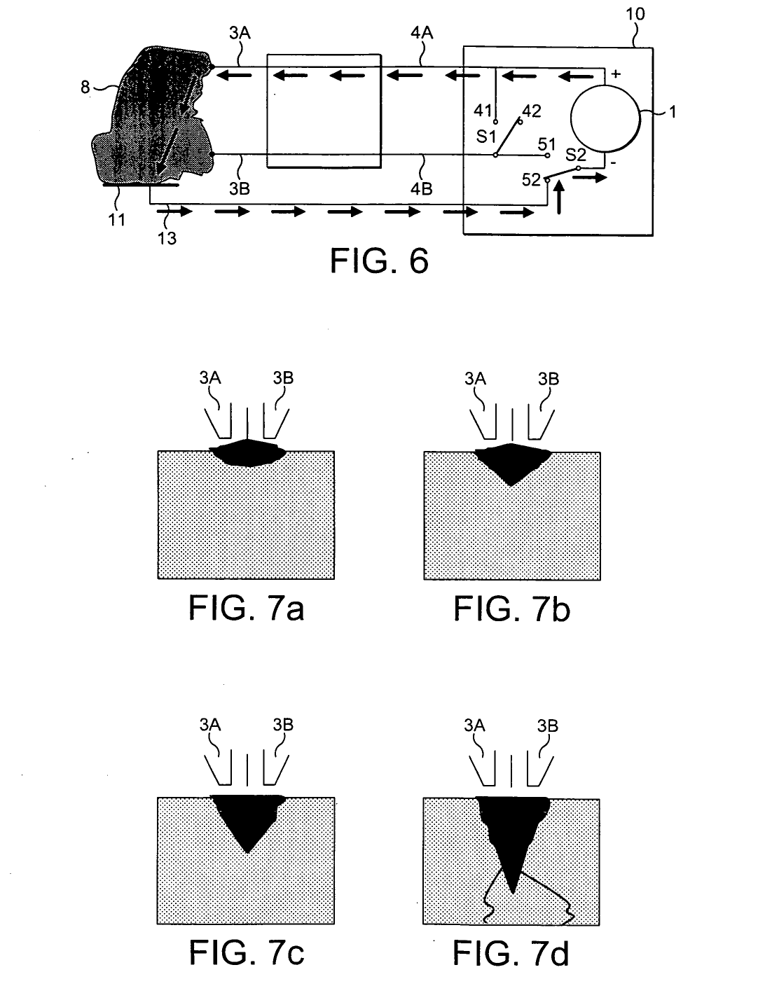

FIG. 6 shows an alternative arrangement in which only bipolar electrode 3A and not electrode 3B is used when the system is in “monopolar” mode. In this embodiment, switch S1 is permanently in its second “open” position 42, or could conceivably be dispensed with. In the blended mode, switch S2 rapidly alternates between its two positions 51 and 52, directing the RF waveform from the power source 1 to between the electrode 3A and either electrode 3B or (as shown in FIG. 6) the patient return plate 11. This is a simpler switching arrangement, but as only one of the two bipolar electrodes is energized in “monopolar” mode, the tissue effect achieved may be more limited to the area surrounding electrode 3A.

FIGS. 7a to 7d shown the tissue effect achieved in the tissue 8 in the region of the electrodes 3A and 3B using different proportions of bipolar and monopolar energy. FIG. 7a shows the effect of using solely the electrodes 3A and 3B in bipolar mode, with tightly controlled and relatively shallow tissue coagulation. This would be used when it is necessary to avoid the unwanted coagulation of sensitive tissue or organs located close to the region where the coagulation is desired. FIG. 7b shows the tissue effect achieved by the embodiment described in FIGS. 1 to 6 above, in which the switches S1 and S2 are controlled such that the system spends more time in each cycle in the bipolar mode (the first supply state) than in the monopolar mode (the second supply state). The tissue effect is slightly deeper, but still relatively shallow. FIG. 7c shows the opposite arrangement in which the switches are controlled such that the system spends more time in each cycle in the monopolar mode as compared with the bipolar mode. In this arrangement, the tissue effect is deeper still. Finally, FIG. 7d shows the system used solely in monopolar mode. In this arrangement, the coagulating effect spreads away from the electrodes 3A and 3B towards the patient return plate (not shown in FIGS. 7a to 7d).

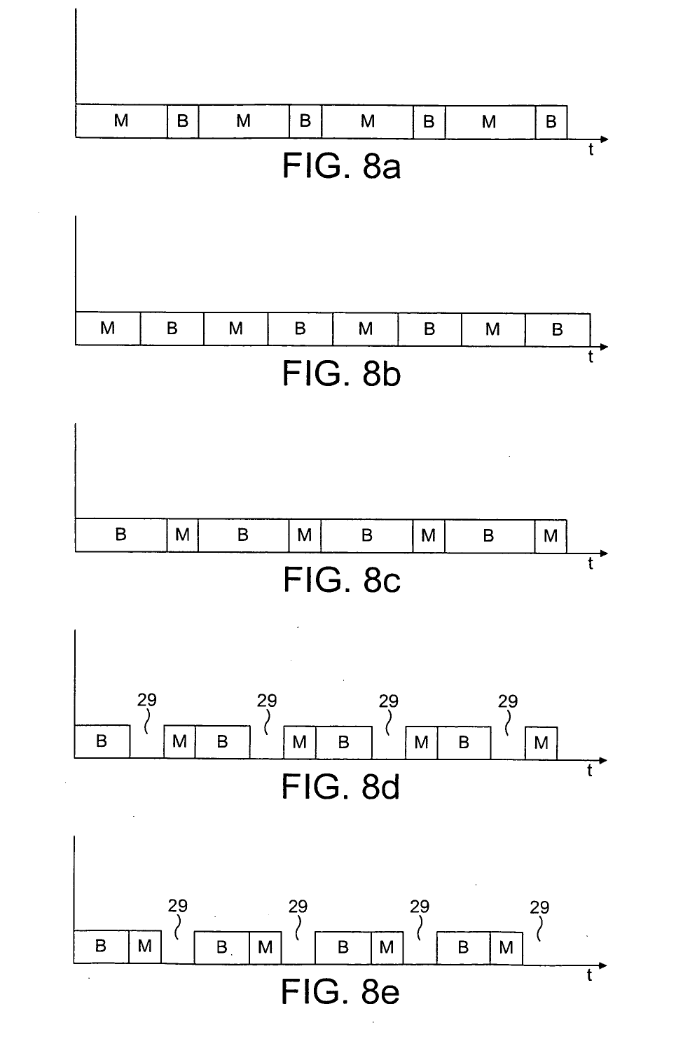

FIGS. 8a to 8e show different arrangements for the timings for the switches S1 and S2. In the figures, the switches are in the positions shown in FIG. 4 for the periods shown as marked with a “B”, indicating the bipolar mode. Conversely, the switches are in the positions shown in FIG. 5 or 6 for the periods shown as marked with an “M”, indicating the monopolar mode. In FIG. 8a, the bipolar mode is approx 25% of the duty cycle (with the monopolar mode making up the remaining 75%). Thus the tissue effect will be much more influenced by the monopolar waveform, and this is the situation depicted in FIG. 7c. In FIG. 8b the first and second duty cycles are both 50%, with energy being delivered equally in the bipolar and monopolar modes. FIG. 8c shows a first duty cycle of 75%, with energy being delivered in the bipolar mode during 75% of each cycle. This is the situation depicted in FIG. 7b, with the bipolar tissue effect being more evident.

In FIGS. 8a to 8c the switches S1 and S2 operate in unison, so that the bipolar mode takes over from the monopolar mode without an interruption, and vice versa. Thus the bipolar and monopolar signals are supplied consecutively to the tissue 8, without a break. Thus when the first duty cycle is 25% the second is 75%, and vice versa. In FIGS. 8d and 8e a deliberate time gap 29 is left between the signals. Referring to FIG. 8d, a gap 29 is left after each bipolar signal, while in FIG. 8e a gap 29 is left after each monopolar signal. Clearly, with the gaps of FIGS. 8d and 8e, the first and second duty cycles do not total 100%. In FIG. 8d, the first duty cycle is 50%, and the second duty cycle 25% (meaning that the gap 29 constitutes 25% of the overall cycle time). In FIG. 8e, the first duty cycle is still 50% and the second is still 25% (the only difference being that the gap 29 comes after the monopolar mode rather than before it).

FIG. 9 shows a variation on FIG. 3, showing an additional switch S3 to produce the gaps 29. Switch S3 has two positions 61 and 62. When switch S3 is in position 61, power from the source 1 is interrupted and does not reach any of the electrodes, producing gaps 29. When switch S3 is in position 62, the power source 1 is connected, and the supply of energy to the electrodes is governed by the position of switches S1 and S2, as previously described.

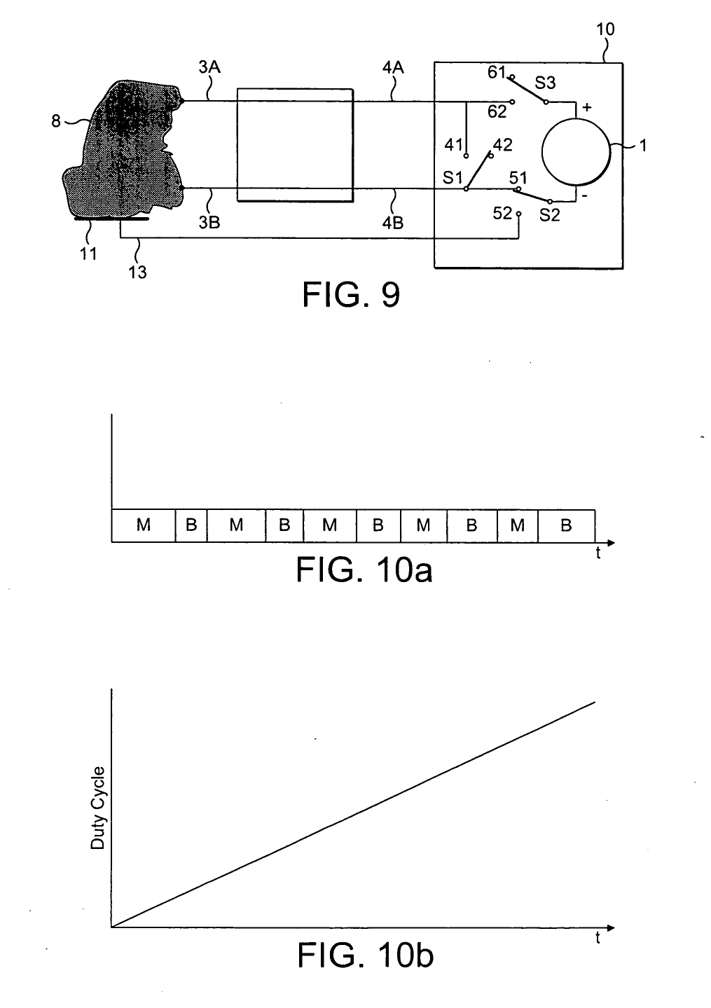

In FIGS. 8a to 8e the duty cycle is constant for one time period as compared with another. However, this does not necessarily need to be the case and FIGS. 10a and 10b show one arrangement in which the first and second duty cycles vary with time. FIG. 10a shows how the first duty cycle starts at 33%, with the second duty cycle being 67% so that the system spends the majority of each cycle in the monopolar mode. As time progresses, the proportion of each cycle spent in the bipolar mode increases, and the proportion of each cycle spent in the monopolar mode decreases. Thus the first duty cycle changes over time from 33% to 67%, in the example shown in FIG. 10a. Clearly the transition will occur in practice over many more cycles than is shown in FIG. 10a, which is for illustrative purposes only. FIG. 10b shows how this can be depicted schematically, with the first duty cycle shown as varying with time. With a low first duty cycle, the proportion of time spent in the bipolar mode is relatively small, and the signal produced is predominantly monopolar. With a higher first duty cycle, the proportion of time spent in the bipolar mode is higher, and the signal produced is predominantly bipolar.

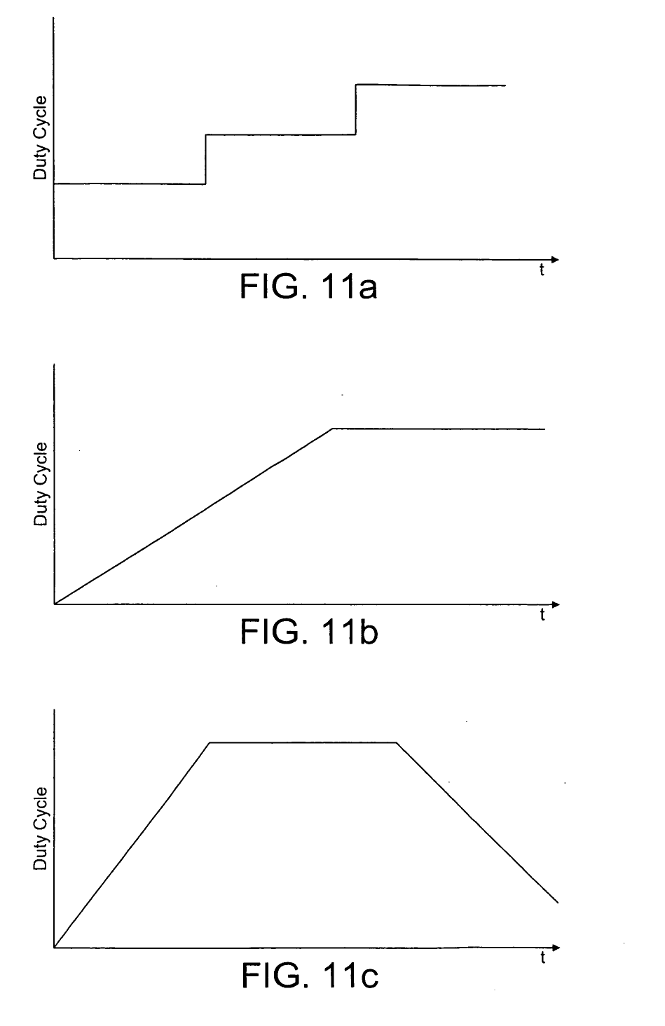

FIGS. 11a to 11c show schematic diagrams, similar to that of FIG. 10b, showing other embodiments of the invention in which the first duty cycle varies. In FIG. 11a, the first duty cycle varies in a stepped fashion, with the changes between different values for the first duty cycle being in discrete steps. The steps could be steadily up (as shown in FIG. 11a) or alternatively steadily down, or some combination of up then down (or vice versa). FIG. 11b shows an arrangement in which the first duty cycle increases in a ramped fashion until a predetermined maximum is reached, in which case the first duty cycle is held constant at a certain value. FIG. 11c shows an arrangement in which the first duty cycle increases in a ramped fashion, is held constant for a predetermined period, and then is ramped down again. This would have the effect of providing a predominantly monopolar tissue effect at the start of treatment, changing to a predominantly bipolar tissue effect in the middle of the treatment, and ending once again with a predominantly monopolar tissue effect. Other progressive or stepped arrangements can clearly be envisioned by those skilled in the art, and may be appropriate for different tissue types or different surgical procedures. Clearly, there is the possibility to vary the second duty cycle instead of the first duty cycle, or both duty cycles where there is the possibility to vary both duty cycles independently.

The arrangements of FIGS. 8, 10 and 11 are fixed or preset progressions. However, any duty cycle can be adaptively controlled based on a parameter associated with the electrosurgical procedure, such as the tissue impedance. As previously described, if the electrosurgical system detects a low tissue impedance (indicating a relatively fluid surgical environment associated with bleeding tissue), the first duty cycle would be adjusted downwardly to increase the proportion of monopolar signal applied to the tissue. Conversely, if the electrosurgical system detects a relatively high tissue impedance (indicating a relatively dry surgical environment), the first duty cycle would be adjusted upwardly to increase the proportion of bipolar signal applied to the tissue. Thus the electrosurgical system can adapt automatically to changes in the surgical environment, without the need for a manual adjustment of the generator by the surgeon.

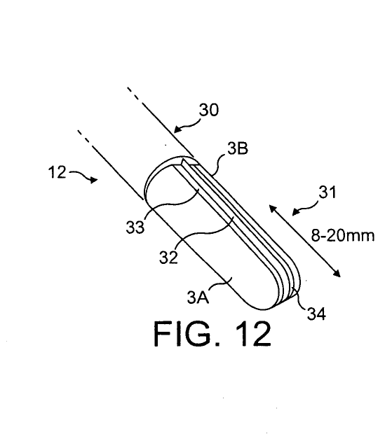

FIG. 12 shows one possible design for the electrosurgical instrument 12. The instrument 12 comprises an instrument shaft 30 at the distal end of which is an electrode assembly shown generally at 31. The electrode assembly 31 comprises a central cutting electrode 32 disposed between two larger coagulation electrodes 3A and 3B. Insulating layer 33 separates the cutting electrode 32 from the first coagulating electrode 3A while insulating layer 34 separates the cutting electrode from the second coagulating electrode 3B. The cutting electrode 32 protrudes slightly beyond the two coagulating electrodes.

When the user intends the instrument to coagulate tissue, the electrosurgical generator supplies an RF waveform between the electrodes 3A and 3B as well as the patient return plate (not shown in FIG. 11) as previously described. When the user intends the instrument to cut tissue, the generator applies a cutting RF waveform between the cutting electrode 32 and one or both of the coagulating electrodes 3A and 3B. The protruding nature of the cutting electrode 32 helps to provide a cutting action when the electrode 32 is brought into contact with tissue.

Those skilled in the art will appreciate that variations on the precise examples given herein can be made without departing from the scope of the present invention. For example, a range of different arrangements for varying the duty cycle, in addition to those described herein, could be readily derived depending on the tissue to be treated, the surgical procedure under consideration, or even the particular preference of each individual surgeon. Any of the embodiments discussed herein can be employed with or without an additional cutting electrode.

1. An electrosurgical system including a generator for generating radio frequency (RF) power, an electrosurgical instrument including at least first and second bipolar electrodes carried on the instrument, and a monopolar patient return electrode separate from the instrument,
wherein the generator comprises at least one source of RF power and a plurality of outputs connected to the electrodes, the generator being adapted to operate in a first supply state in which an RF output waveform is delivered between the first and second bipolar electrodes via the output lines, and in a second supply state in which an RF output waveform is delivered between (a) at least one of the first and second bipolar electrodes and (b) the monopolar patient return electrode via the output lines, which operation, in at least one mode of the generator, includes continuously alternating between the first supply state and the second supply state whereby combined bipolar and monopolar RF energy delivery is obtained.

2. An electrosurgical system according to claim 1, wherein a first duty cycle is the proportion of time that the generator operates in the first supply state, and a second duty cycle is the proportion of time that the generator operates in the second supply state.

3. An electrosurgical system according to claim 2, wherein the generator comprises feeding means arranged to cause the generator to operate such that the first and second duty cycles are both 50%.

4. An electrosurgical system according to claim 2, further comprising adjustment means, operable by a user of the electrosurgical system, for changing at least one duty cycle.

5. An electrosurgical system according to claim 4 wherein the adjustment means is operable by the user of the electrosurgical system to change at least one duty cycle between a plurality of preset settings.

6. An electrosurgical system according to claim 4, further comprising means for measuring a parameter associated with the electrosurgical procedure, the controller adjusting at least one duty cycle automatically in response to the measured parameter.

7. An electrosurgical system according to claim 6, wherein the measured parameter is the impedance measured across two of the electrodes.

8. An electrosurgical system according to claim 3, wherein the feeding means operates such that at least one duty cycle varies according to a predetermined progression.

9. An electrosurgical system according to claim 8, wherein the predetermined progression is such that at least one duty cycle increases with time.

10. An electrosurgical system according to claim 8, wherein the predetermined progression is such that at least one duty cycle decreases with time.

11. An electrosurgical system according to claim 8, wherein the feeding means operates such that there is a first period during which both duty cycles are constant, followed by a second period in which at least one duty cycle varies according to a predetermined progression.

12. An electrosurgical system according to claim 2, wherein the first and second duty cycles are such that there are gaps between successive operation in at least one of the first and second supply states.

13. An electrosurgical system according to claim 11, wherein the first and second duty cycles are such that there are gaps between successive operation in at least one of the first and second supply states.

14. An electrosurgical system according to claim 2, wherein a characteristic of the RF output waveform is associated with the first duty cycle is different as compared to that characteristic of the RF output waveform associated with the second duty cycle.

15. An electrosurgical system according to claim 13, wherein a characteristic of the RF output waveform is associated with the first duty cycle is different as compared to that characteristic of the RF output waveform associated with the second duty cycle.

16. An electrosurgical system according to claim 14, wherein the characteristic is selected from the power of the RF waveforms, the voltage of the RF waveforms, the current of the RF waveforms, and the frequency of the RF waveforms.

17. An electrosurgical system according to claim 15, wherein the characteristic is selected from the power of the RF waveforms, the voltage of the RF waveforms, the current of the RF waveforms, and the frequency of the RF waveforms.

18. An electrosurgical system according to claim 17, wherein the electrosurgical instrument includes at least a third electrode, and the generator is adapted, in an alternative mode of operation, to supply a cutting RF waveform between the third electrode and at least one of the first and second electrodes.

19. An electrosurgical system according to claim 16, wherein the electrosurgical instrument includes at least a third electrode, and the generator is adapted, in an alternative mode of operation, to supply a cutting RF waveform between the third electrode and at least one of the first and second electrodes.

20. An electrosurgical generator for generating radio frequency (RF) power, wherein the generator comprises a bipolar output having at least two output lines for coupling to bipolar electrodes of a bipolar electrosurgical instrument, and a monopolar output having at least one output line for a monopolar patient return electrode separate from the instrument, wherein the generator comprises at least one source of RF power and is adapted to operate in a first supply state in which an RF output waveform is delivered between the two output lines of the bipolar output, and a second supply state in which an RF output waveform is delivered between (a) at least one of the two output lines of the bipolar output and (b) the output line of the monopolar output, and wherein the generator further comprises a controller operable in at least one mode of the generator, to cause operation of the generator to alternate continuously between the first supply state and the second supply state for combined bipolar and monopolar RF energy delivery.
